(19) United States
(12) Patent Application Publication
(10) Pub. No.: US 2023/0337960 A1
(43) Pub. Date: Oct. 26, 2023

(54) PROJECTING ACTIVATION WAVE VELOCITY ONTO MAPPED CARDIAC CHAMBER

(71) Applicant: BIOSENSE WEBSTER (ISRAEL) LTD., Yokneam (IL)

(72) Inventor: Assaf Govari, Haifa (IL)

(21) Appl. No.: 17/724,677

(22) Filed: Apr. 20, 2022

(51) Int. Cl. A61B 5/367; A61B 5/343; A61B 5/00

(52) U.S. Cl. CPC A61B 5/367; A61B 5/343; A61B 5/7221; A61B 5/7475

(57) ABSTRACT

A method includes receiving an electrophysiological (EP) map of a cardiac chamber, the EP map including data points comprising respective locations and EP values. The EP map is projected onto a sphere divided into a grid of unit areas. For at least some of the unit areas, a most likely data point is estimated that is representative of an EP activation in the unit area. The representative data points is inverse mapped onto the EP map. An updated EP map with the inverse mapped representative data points is presented to a user.

### FIELD OF THE DISCLOSURE

This disclosure relates generally to electrophysiological (EP) mapping, and specifically to a method for improving accuracy of cardiac EP maps.

### BACKGROUND OF THE DISCLOSURE

Methods of cardiac mapping were previously described in the patent literature. For example, U.S. Pat. No. 10,136,828 describes electroanatomic mapping that is carried out by inserting a multi-electrode probe into a heart of a living subject, recording electrograms from the electrodes concurrently at respective locations in the heart, delimiting respective activation time intervals in the electrograms, generating a map of electrical propagation waves from the activation time intervals, maximizing coherence of the waves by adjusting local activation times within the activation time intervals of the electrograms, and reporting the adjusted local activation times.

As another example, U.S. Pat. No. 10,198,876 describes systems and methods for visualizing and analyzing cardiac arrhythmias using 2-D planar projection and partially unfolded surface mapping processes. A method for projecting a 3D surface geometry onto a planar projection comprises: obtaining a 3D geometry of a chamber surface using an algorithm that generates angles and distances between points on the chamber surface that represent mapping information; applying a cutting curve to at least two points on the chamber surface; and at least partially unfolding at least a portion of the chamber surface along the cutting curve to form a planar projection that optimally preserves the angles and distances between points on the chamber surface.

### BRIEF DESCRIPTION OF THE DRAWINGS

The present disclosure will be more fully understood from the following detailed description of the examples thereof, taken together with the drawings, in which:

FIG. 1 is a schematic, pictorial illustration of a catheter-based electrophysiological (EP) mapping system, according to an example of the present disclosure;

FIG. 2 is a schematic, pictorial illustration of a 2D sphere model of an EP map of a left atrium (LA), according to an example of the present disclosure;

FIG. 3 is a schematic, pictorial illustration of inverse mapping of a statistically determined EP value from the 2D sphere model of FIG. 2 to the EP map of the left atrium (LA), according to an example of the present disclosure; and

FIG. 4 is a flow chart schematically describing a method of correcting the EP map of FIG. 3 using statistical analysis over the 2D sphere model of FIG. 2, according to an example of the present disclosure.

### DETAILED DESCRIPTION OF EXAMPLES

### Overview

Electrophysiological (EP) cardiac mapping is often used for identifying potential sources of cardiac arrhythmia in cardiac tissue. Physicians typically use a commonly known coherent type of EP mapping to visualize a global pattern of activation in a chamber of interest. In the context of the present description, the term “coherent” means an EP activation wave propagation description, in which conduction velocity is both cyclic and continuous. To this end, a mapping system may use catheter-acquired anatomical locations and local activation times (LAT) acquired at the respective locations to build such a coherent map. The coherent map includes arrows that show the magnitude and direction of activation waves. Physicians may use other EP map types to characterize arrhythmia, such as bipolar potential maps.

Regardless of the EP map type, various noises during catheter acquisition may produce inaccuracies of the EP map at specific locations over a cardiac chamber. A physician may therefore want to sample more data points (e.g., locations and EP signals related to these locations) in order to increase the accuracy of the EP map in specific areas of the cardiac chamber. In addition, the physician often wants to reexamine the pattern of activation after an ablation procedure in order to check if the ablation has succeeded in cutting off aberrant transfer of the activation wave across a region of interest (ROI), which typically requires the physician to remap the signals.

In some examples of the present disclosure that are described herein, more than one data point is collected at particular locations, e.g., the acquired data points are temporally scattered. A disclosed method uses the collection of data points at a particular area to improve the accuracy of the EP map. For example, as the signals are acquired by the catheter, a processor may store the calculated magnitude and direction values for each point in each area. The accumulation of data for each such area may provide corrections and/or improvements to the map in these areas, which can be used for local correction or updating of the map.

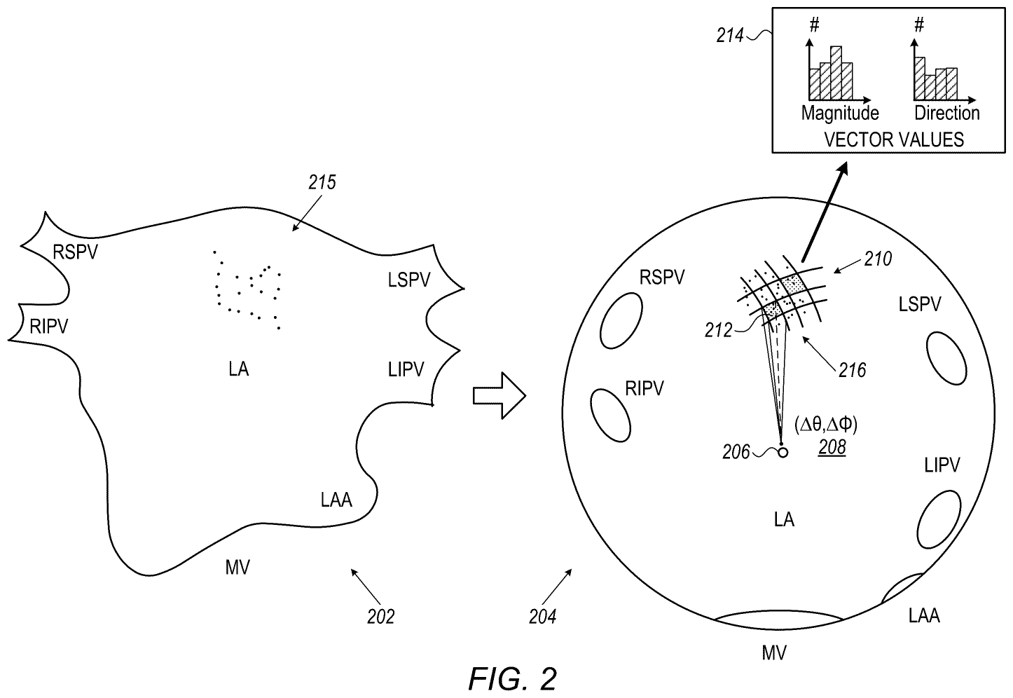

In one example, the processor models the anatomy of the cardiac chamber as a sphere having a grid for example, performs mapping (e.g., projection) of the EP map anatomy onto a sphere, or (ϕ,θ) mapping of the anatomy. The projection results in a surface area on the sphere which can be defined with 2D coordinates (θ,ϕ). The 2D mapping is possible because the EP map is typically not associated with a thickness. The processor divides the 2D surface into unit areas according to the grid (one possible selection being (Δθ,Δϕ)) patches). For a given area, the processor saves the data points collected in that area during the EP mapping procedure, and relates that data to the particular location. The analysis over a 2D sphere model is much more efficient than on the EP map itself, and, as a result, an improved map can be generated (and refreshed) in real time, for example by inverse-mapping (e.g., back projecting) of the sphere data points onto the EP map anatomy using the disclosed technique, as described in FIG. 2 below.

In some examples, the processor determines a statistically robust representative EP value for each unit area based on the multiple data points acquired for that area. The processor uses a suitable optimization algorithm (e.g., a genetic algorithm or a Wisdom of Crowd (WoC) algorithm) for this purpose. In some examples, a confidence level layer is added, indicating to a physician how robust the EP map is in each location. The confidence level may indicate, for example, the probability with which the statistically determined representative EP value is true.

Moreover, the processor can also use the statistically determined location itself with a unit area to locally correct the anatomical shape of the EP map.

Typically, the processor is programmed in software containing a particular algorithm that enables the processor to conduct each of the processor related steps and functions outlined above.

### SYSTEM DESCRIPTION

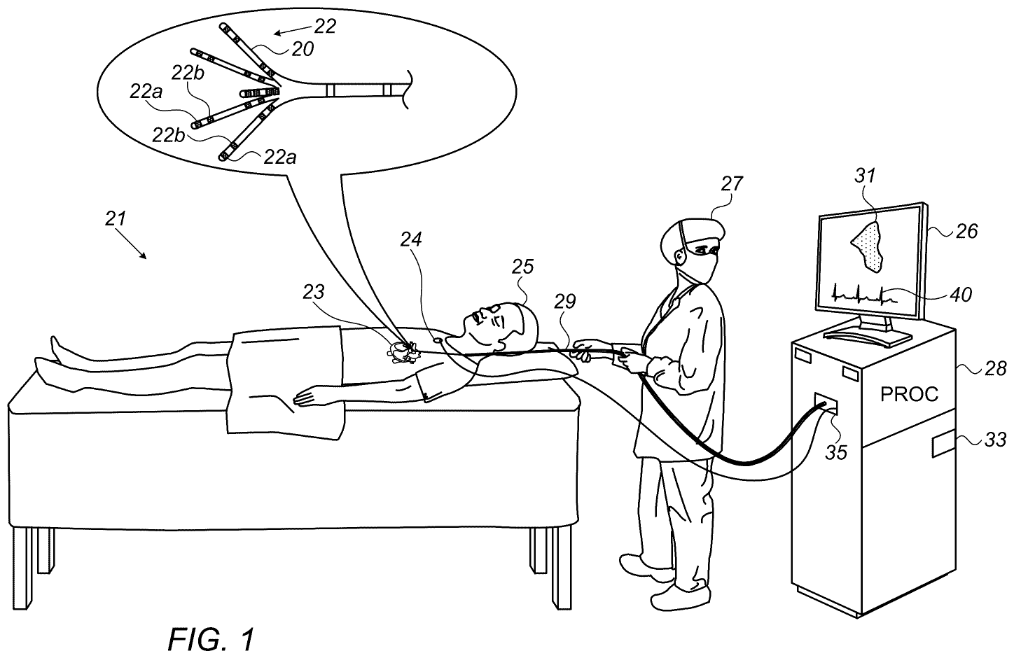

FIG. 1 is a schematic, pictorial illustration of a catheter-based electrophysiological (EP) mapping system 21, according to an example of the present disclosure. FIG. 1 depicts a physician 27 using an electro-anatomical mapping catheter 29 to perform an electro-anatomical mapping of a heart 23 of a patient 25. Mapping catheter 29 comprises, at its distal end, one or more arms 20, each of which is coupled to a bipolar electrode 22 comprising adjacent electrodes 22a and 22b. Any of electrodes 22 can be used in a unipolar acquisition mode by, for example, acquiring an intracardiac potential relative to an external electrode 24 attached to chest skin of patient 25.

During the mapping procedure, the locations of electrodes 22 are tracked while they are inside heart 23 of the patient. For that purpose, electrical signals are passed between electrodes 22 and external electrodes 24. For example, three external electrodes 24 may be coupled to the patient's chest, and another three external electrodes may be coupled to the patient's back. For ease of illustration, only one external electrode is shown in FIG. 1. In some examples, the distal end of the catheter includes a magnetic sensor (not shown) that allows to magnetically track the locations of electrodes 22 or to calibrate the aforementioned the electrical tracking signals to improve an accuracy of an electrical location tracking method.

Based on these signals, and given the known positions of electrodes 24 on the patient's body, processor 28 calculates an estimated location of each electrode 22 within the patient's heart. Respective electrophysiological data, such as bipolar electrogram traces, are additionally acquired from tissue of heart 23 by using electrodes 22. The processor 28 may thus associate any given signal received from electrodes 22, such as a bipolar EP signal, with the location at which the signal was acquired. The processor 28 receives the resulting signals via an electrical interface 35, and uses information contained in these signals to construct an electrophysiological map 31 and EGM or ECG traces 40, and to present these on a display 26. The EGM or ECG traces 40 are also stored in a memory 33 for use with the disclosed algorithm. One tracking system and method capable of producing map 31 is the Advanced Current Location (ACL) system, implemented in various medical applications, for example, in the CARTO™ system, produced by Biosense-Webster Inc., which is described in detail in U.S. Pat. No. 8,456,182 whose disclosure is incorporated herein by reference. A magnetic position tracking system (not shown) and method capable of producing map 31 or improving the accuracy of the ACL method is described in U.S. Pat. Nos. 5,391,199, 6,690,963, 6,484,118, 6,239,724, 6,618,612 and 6,332,089, in PCT Patent Publication WO 96/05768, and in U.S. Patent Application Publication Nos. 2002/0065455 A1, 2003/0120150 A1 and 2004/0068178 A1.

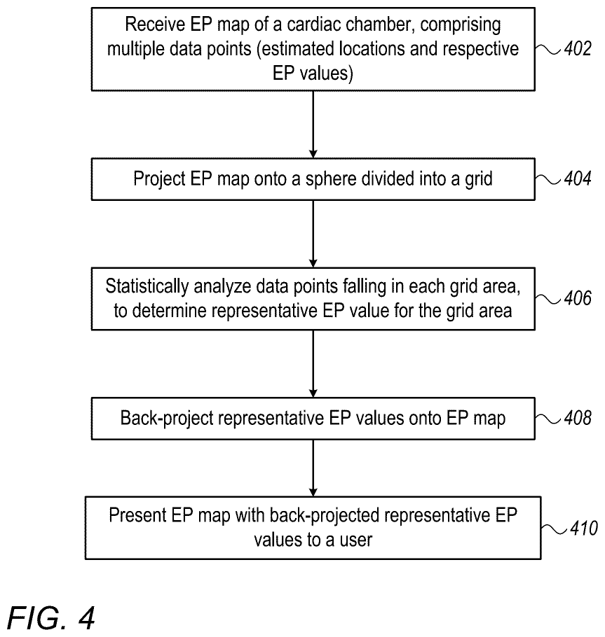

Processor 28 typically comprises a general-purpose computer with software programmed to carry out the functions described herein. The software may be downloaded to the computer in electronic form, over a network, for example, or it may, alternatively or additionally, be provided and/or stored on non-transitory tangible media, such as magnetic, optical, or electronic memory. In particular, processor 28 runs a dedicated algorithm as disclosed herein, including in FIG. 4, that enables processor 28 to perform the disclosed steps, as further described below.

The example illustration shown in FIG. 1 is chosen purely for the sake of conceptual clarity. Other types of electrophysiological sensing catheter geometries, such as the Lasso® Catheter (produced by Biosense-Webster Inc., Irvine, California) may be employed. Additionally, contact sensors may be fitted at the distal end of mapping catheter 29 to transmit data indicative of the physical quality of electrode contact with tissue. In an example, measurements of one or more electrodes 22 may be discarded if their physical contact quality is indicated as poor, and the measurements of other electrodes may be regarded as valid if their contact quality is indicated as sufficient.

2D Modeling of an Ep Map of a Cardiac Chamber

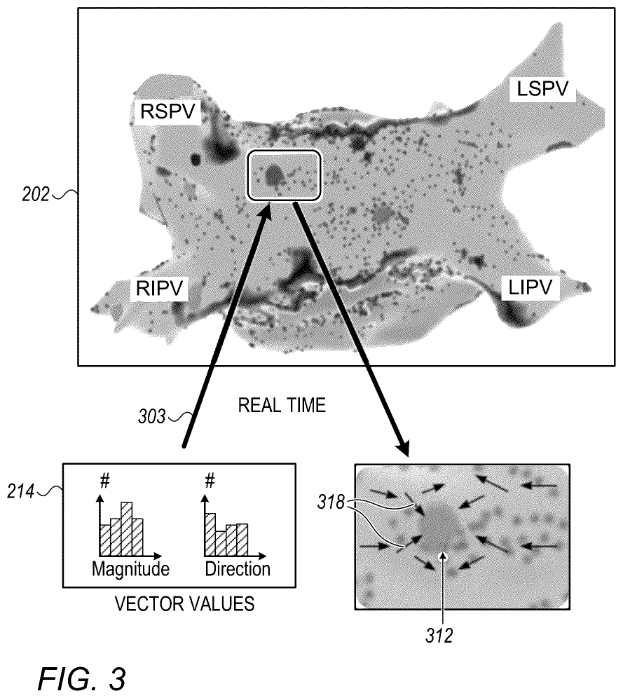

FIG. 2 is a schematic, pictorial illustration of a 2D sphere model 204 of an EP map 202 of a left atrium (LA), according to an example of the present disclosure. As seen, the LA has features comprising four pulmonary veins (PV), a left atrial appendage (LAA) and mitral valve (MV). The acquisition of data points in the LA may be challenging, leading to local inaccuracies in EP map 202, as discussed above. Still, a chamber shape is simple enough to be (ϕ,θ) mapped onto sphere 204, and, using the statistical analysis on the sphere, the disclosed technique can improve the accuracy of map 202, as described below and in FIG. 3.

As seen in FIG. 2, sphere 204 has a spherical grid 210 defined by angular sections 208 from an origin 206 of sphere 204. Each angular section 208 corresponds to a unit area 212, and the size of areas 212 can be predefined or updated according to, for example, required spatial resolution or robustness of statistical corrections to EP map 202. Areas 212 typically measure several mm².

As intended by the disclosed technique to improve EP map accuracy, additional acquisitions by the catheter provide multiple data points 216 in same areas 212 of sphere 204, particularly in demanding locations 215 of the cardiac chamber. The data points 216 acquired may be for example magnitude and direction of the detected activation. In some example embodiments, processor 28 may evaluate the data points acquired in an area 212 over time and select the most likely data point that is representative of the activation in that area. Alternatively or additionally, processor may evaluate the data points acquired in an area 212 and identify outliers based comparing data points with an identified representative data point. Optionally, statistical analysis is performed to select the most representative data point. Optionally, a Wisdom of Crowd algorithm is used for this purpose. The processor then applies statistical analysis to data points 216 in each area 212 to statistically determine a representative EP value therein. An example of the statistical analysis is brought in the form of activation velocity histograms 214, in case the EP value sought is a local activation velocity. There can be between 10-100 or more points per unit area.

The output of the wisdom of crowd algorithm is a single representative point per unit area. The processor can compute for every unit area a magnitude and direction of the EP propagation signal of the heart.

FIG. 3 is a schematic, pictorial illustration of inverse mapping (303) of a statistically determined representative EP value from the 2D sphere model 204 of FIG. 2 to the EP map 202 of the left atrium (LA), according to an example of the present disclosure. In the context of this disclosure, an inverse mapping is a mathematical operation, such as back projection, or other conformal transformation, of a set of data points overlaid on a sphere into an anatomical surface from which they were projected onto the sphere.

As seen, there is a need to make the EP map more accurate in an area 312 of the map, which corresponds to one or more areas 212 on sphere 202. Specifically, there is a need to know more accurately the activation velocity therein, to, for example, determine whether or not the area is arrhythmogenic. Using the disclosed technique, the processor inverse-maps (303) the analysis-results from areas 212 of sphere 204 onto area 312 in the form of statistically determined EP values that activation velocities 318 are derived from, which makes the EP map more clinically meaningful in terms of activation wave ways of propagation.

### Method of Correcting an Ep Map Using Statistical Analysis on 2D Sphere Model

FIG. 4 is a flow chart schematically describing a method of correcting EP map 202 of FIG. 3 using statistical analysis over 2D sphere model 204 of FIG. 2, according to an example of the present disclosure. The algorithm according to the presented embodiment carries out a process that begins with processor 28 receiving EP map 202 of a cardiac chamber, the EP map comprising data points comprising respective locations and EP values, at an EP map receiving step 402.

At a (ϕ,θ) mapping step 404, processor 28 projects EP map 202 onto a sphere 204 divided into a grid 210 of unit areas 212 (also called herein “grid areas”).

Next, at a statistical analysis step 406, for at least some of the unit areas, the processor statistically analyzes the data points falling in each unit area to determine a representative EP value for the unit area (e.g., grid area).

At an inverse mapping step 408, the processor inverse maps (e.g., back-projects) the representative EP values onto EP map 202.

Finally, at EP map presentation step 410, processor 28 presents EP map 202 with the inversed mapped representative EP values to a user.

The flow chart of FIG. 4 is brought by way of example.

As another example, different grid systems may be used for the sphere model.

### EXAMPLES

### Example 1

A method includes receiving an electrophysiological (EP) map (202) of a cardiac chamber, the EP map including data points comprising respective locations and EP values. The EP map is projected onto a sphere (204) divided into a grid (210) of unit areas (212). For at least some of the unit areas (212), a most likely data point is estimated that is representative of an EP activation in the unit area. The representative data points is inverse mapped onto the EP map (202). An updated EP map with the inverse mapped representative data points is presented to a user.

### Example 2

The method according to example 1, wherein estimating a most likely data point comprises using a Wisdom of Crowd (WoC) algorithm.

### Example 3

The method according to any of claims 1 and 2, wherein the grid is a spherical grid.

### Example 4

The method according to any of examples 1 through 3, and comprising receiving additional data points and projecting the additional data points onto the grid (210). The data points and the additional data points are estimated to re-determine the most likely data point that are representative of the activations for one or more of the unit areas. The re-determined representative data points are inverse mapped, and the updated EP map view presented to the user is refreshed to reflect the re-determined representative data points.

### Example 5

The method according to any of examples 1 through 4, and comprising calculating and presenting to the user levels of confidence associated with one or more of the representative data points.

### Example 6

The method according to any of examples 1 through 5, wherein the data points comprise local activation velocities.

### Example 7

The method according to any of examples 1 through 6, wherein the data points comprise local activation times (LAT) of an activation wave.

### Example 8

The method according to any of examples 1 through 7, wherein the data points comprise local potentials.

### Example 9

A system includes an interface (35) and a processor (28). The interface (35) is configured to receive an electrophysiological (EP) map (202) of a cardiac chamber, the EP map comprising data points comprising respective locations and EP values. The processor (28) is configured to (i) project the EP map (202) onto a sphere (204) divided into a grid (210) of unit areas (212), (ii) for at least some of the unit areas, estimate a most likely data point that is representative of an EP activation in the unit area, (iii) inverse map the representative data points onto the EP map, and (iv) present an updated EP map with the inverse mapped representative data points to a user.

Although the examples described herein mainly address cardiac diagnostic applications, the methods and systems described herein can also be used in other medical applications.

It will be appreciated that the examples described above are cited by way of example, and that the present disclosure is not limited to what has been particularly shown and described hereinabove. Rather, the scope of the present disclosure includes both combinations and sub combinations of the various features described hereinabove, as well as variations and modifications thereof which would occur to persons skilled in the art upon reading the foregoing description and which are not disclosed in the prior art.

1. A method, comprising:
receiving an electrophysiological (EP) map of a cardiac chamber, the EP map comprising data points comprising respective locations and EP values;
projecting the EP map onto a sphere divided into a grid of unit areas;
for at least some of the unit areas, estimating a most likely data point that is representative of an EP activation in the unit area;
inverse mapping the representative data points onto the EP map; and
presenting an updated EP map with the inverse mapped representative data points to a user.

2. The method according to claim 1, wherein estimating a most likely data point comprises using a Wisdom of Crowd (WoC) algorithm.

3. The method according to claim 1, wherein the grid is a spherical grid.

4. The method according to claim 1, and comprising receiving additional data points, projecting the additional data points onto the grid, estimating the data points and the additional data points to re-determine the most likely data point that are representative of the activations for one or more of the unit areas, inverse mapping the re-determined representative data points, and refreshing the updated EP map view presented to the user to reflect the re-determined representative data points.

5. The method according to claim 1, and comprising calculating and presenting to the user levels of confidence associated with one or more of the representative data points.

6. The method according to claim 1, wherein the data points comprise local activation velocities.

7. The method according to claim 1, wherein the data points comprise local activation times (LAT) of an activation wave.

8. The method according to claim 1, wherein the data points comprise local potentials.

9. A system, comprising:
an interface configured to receive an electrophysiological (EP) map of a cardiac chamber, the EP map comprising data points comprising respective locations and EP values; and
a processor, which is configured to:
project the EP map onto a sphere divided into a grid of unit areas;
for at least some of the unit areas, estimate a most likely data point that is representative of an EP activation in the unit area;
inverse map the representative data points onto the EP map; and
present an updated EP map with the inverse mapped representative data points to a user.

10. The system according to claim 9, wherein the processor is configured to estimate a most likely data point by using a Wisdom of Crowd (WoC) algorithm.

11. The system according to claim 9, wherein the grid is a spherical grid.

12. The system according to claim 9, wherein the interface is further configured to receive additional data points, and wherein the processor is further configured to project the additional data points onto the grid, estimate the data points and the additional data points to re-determine the most likely data point that are representative of the activations for one or more of the unit areas, inverse map the re-determined representative data points, and refresh the updated EP map view presented to the user to reflect the re-determined representative data points.

13. The system according to claim 9, wherein the processor is further configured to calculate and presenting to the user levels of confidence associated with one or more of the representative data points.

14. The system according to claim 9, wherein the data points comprise local activation velocities.

15. The system according to claim 9, wherein the data points comprise local activation times (LAT) of an activation wave.

16. The system according to claim 9, wherein the data points comprise local potentials.
